(19) United States
(12) Patent Application Publication
(10) Pub. No.: US 2008/0154512 A1
(43) Pub. Date: Jun. 26, 2008

(54) SYSTEMS AND METHODS FOR BASELINING AND REAL-TIME PCR DATA ANALYSIS

(76) Inventor: Harrison Leong, San Francisco, CA (US)

Correspondence Address: MH2 TECHNOLOGY LAW GROUP, LLP, 1951 KIDWELL DRIVE, SUITE 550, TYSONS CORNER, VA, 22182, US

(21) Appl. No.: 11/923,633

(22) Filed: Oct. 24, 2007

Related U.S. Application Data
(63) Continuation-in-part of application No. 11/428,384, filed on Jul. 1, 2006.
(60) Provisional application No. 60/862,729, filed on Oct. 24, 2006.

(51) Int. Cl. C12Q 1/68; G01N 33/48; G06F 19/00

(52) U.S. Cl. 702/19; 435/6; 702/189

(57) ABSTRACT

Systems and methods according to embodiments of the present teachings incorporate a set of possible signal transforms that can be used to examine the baseline region of an amplification profile for noise. In embodiments, a difference time series analysis can be performed to determine deviations of detected fluorescent or other signal intensity in the early cycles of a PCR or other reaction over a median difference time series magnitude. In embodiments, difference time series analysis or other detection techniques can be performed over different hop sizes producing a multi-resolution analysis. In embodiments, the amplification profile can be transmitted to a set of noise detectors whose individual results or decisions are polled or weighted to determine the presence of noise in the baseline or other region. In embodiments, a second derivative analysis on the baseline region can be performed.

### CROSS-REFERENCE TO RELATED APPLICATIONS

This application claims priority to, and is a continuation-in-part of, copending U.S. application Ser. No. 11/428,384, filed Jul. 1, 2006, entitled “Automated Ct Extraction from Amplification Data” by Harrison Leong, which application is assigned to the assignee of the present application, and which application is incorporated by reference herein in its entirety; this application also claims priority to U.S. Provisional Application No. 60/862,729, filed Oct. 24, 2006, entitled “Method for Baselining and Real-time PCR Data Analysis” by Harrison Leong, which provisional application is assigned to the assignee of the present application, and which provisional application is incorporated herein by reference in its entirety.

### INTRODUCTION

Quantitative nucleic acid analysis is extensively used in biological research and clinical analysis. Some of the applications which make use of this technology include: measurement of gene expression, monitoring of biological responses to stimuli, genomic-level gene quantitation, and pathogen detection. Typically, these methodologies utilize Polymerase Chain Reaction (PCR) as a means for selectively amplifying nucleic acid sequences in a manner that allows for their detection.

While it is generally desirable to automate the quantitation process, conventional methodologies often require a degree of user input in the form of subjective interpretation and/or approximation. As a result, these techniques may suffer from reduced accuracy and significant user-induced variability. Furthermore, in high-throughput applications where many samples are to be processed simultaneously, it is desirable to provide increased automation capabilities to improve the speed with which the analysis may be conducted. The aforementioned limitations of conventional techniques illustrate the need for an improved method for analyzing data generated by PCR-based quantitation techniques that may increase the potential for automation while improving the quantitative accuracy and reproducibility of the analysis.

### SUMMARY

Systems and methods according to embodiments of the present teachings incorporate a set of signal transformations that can be used to examine the baseline region or other section of an amplification profile for the presence of noise, exponential growth, or other time series characteristics. In embodiments, an analysis of a difference time series can be performed to determine the location of critical transition regions of real-time PCR data (any data wherein the physical process underlying it results in exponentially increasing signals) and distinguish signals from noise. In embodiments, difference time series analysis can be performed or applied over a set of different step or hop sizes. The baseline or other signals sampled in this fashion can be used to generate difference time series measurements or other computations at multiple levels of resolution over the time series of PCR amplification. In embodiments, the time series of PCR amplification can be analyzed by a set of noise detectors whose individual results or decisions are combined to reach a collective determination regarding the presence or absence of bona fide amplification in the of noise in the baseline or other region. In embodiments, a second derivative analysis on the baseline region can be performed and the criteria used for declaring the onset of growth can be adjusted based on the number or frequency of second derivative peaks.

### BRIEF DESCRIPTION OF DRAWINGS

Systems and methods according to various embodiments of the present teachings will be described with reference to the accompanying drawings, in which like numbers reference like elements, and in which:

FIG. 1 is a schematic illustration of a system for spectral detection and analysis, according to various embodiments of the present teachings;

FIG. 2 is a schematic illustration of a system used for fluorescent signal detection, according to various embodiments of the present teachings;

FIG. 3 is an illustration of an amplification plot or profile depicting reaction characteristics for an exemplary nucleic acid target and various analytical components that can be used to quantify the target, according to various embodiments of the present teachings;

FIGS. 4(A) and 4(B) illustrate signal behavior and difference time series processing, according to various embodiments of the present teachings;

FIGS. 5(A) and 5(B) illustrate signal behavior and multi-step size signal processing, according to various embodiments of the present teachings;

FIGS. 6(A) and 6(B) illustrate signal behavior, noise detector configuration, and noise determination based on multiple detectors, according to various embodiments of the present teachings; and

FIGS. 7(A) and 7(B) illustrate signal behavior and signal processing using second derivative analysis, according to various embodiments of the present teachings.

### DETAILED DESCRIPTION

In accordance with the present teachings, amplification data analysis may be enhanced through signal identification logic and detection routines that make use of transformations of the amplification profile to analyze the noise content, baseline region, exponential onset, and other components or characteristics of the detected signal. In various embodiments, a multi-resolution difference time series approach can be used to determine a baseline interval or other region of interest in an amplification profile as well as detect and localize time series anomalies. In various embodiments, difference time series may be analyzed to identify Runs, i.e., successive data points of the difference time series where selected data points in the Run fulfill selected threshold criteria. In embodiments, the difference time series may be normalized by the median of the absolute value of this time series. In embodiments, deviations of the difference time series from the median of the difference time series within the baseline interval may be used.

In embodiments, further noise detection and time series treatment techniques using derivative, median, and other measures can be used to determine whether or not an amplification profile contains exponential signal growth.

In other embodiments, the analytical approaches described herein can include using the number of significant peaks identified within a second derivative of the amplification profile to determine criteria that may be used to verify the presence of signal growth. The stringency of the criteria for this analysis is increased as the number of significant peaks increases.

In embodiments, the analytical method may include the implementation of detection routines for identifying aberrant baselines and provide correction methods for identified aberrant baselines. Such aberrant baselines include, for example, short baselines occurring in the midst of the time series, time series for which some data following the baseline end point occurs on the side of baseline that is generally opposite that of the sense of growth, and time series for which the baseline end point is within the exponential growth region resulting in a baseline region that appears to descend or dip to the opposite side of the baseline before continuing in the growth direction.

Reference will now be made to the drawings wherein like numerals refer to like elements throughout. As used herein, “target”, “target polynucleotide”, and “target sequence” and the like refer to a specific polynucleotide sequence that is the subject of hybridization with a complementary polynucleotide, e.g., a blocking oligomer, or a cDNA first strand synthesis primer. The target sequence can be composed of DNA, RNA, analogs thereof, or combinations thereof. The target can be single-stranded or double-stranded. In primer extension processes, the target polynucleotide which forms a hybridization duplex with the primer may also be referred to as a “template.” A template serves as a pattern for the synthesis of a complementary polynucleotide (Concise Dictionary of Biomedicine and Molecular Biology, (1996) CPL Scientific Publishing Services, CRC Press, Newbury, UK). A target sequence for use with the present teachings may be derived from any living or once living organism, including but not limited to prokaryote, eukaryote, plant, animal, and virus, as well as synthetic and/or recombinant target sequences.

Furthermore, in describing the present teachings, as used herein the polynucleotide sequence may refer to a polynucleotide chain of variable length and may comprise RNA, DNA, cRNA, cDNA, or other polynucleotide species including but not limited to analogs having other than a phosphodiester backbone. Furthermore, as used herein, “reaction interval” refers to a designated portion of a target amplification reaction and may be evaluated as a function of cycle number or reaction time. Additionally, as used herein, “intensity data” refers to a measured or observed signal generated during the amplification reaction which may be related to the amount of target in the reaction and may comprise fluorescent measurements, radiolabel measurements, electrical measurements, light emission measurements, and other types of signals and measurements generated and acquired during the amplification reaction.

In general, amplification of a target DNA strand by polymerase chain reaction (PCR) proceeds through a series of temperature-regulated cycles using the activity of a thermostable enzyme and a sequence specific primer set. At an appropriate temperature, primers hybridize to portions of the DNA strand and the enzyme successively adds a plurality of nucleotide bases to elongate the primer resulting in the production of progeny (daughter) strands. Each progeny strand possesses a complimentary composition relative to the target strand from which it was derived and can serve as a target in subsequent reaction cycles.

When applying quantitative methods to PCR-based technologies, a fluorescent probe or other detectable reporter construct may be incorporated into the reaction to provide a means for determining the progress of the target amplification. In the case of a fluorescent probe, the reaction can be made to fluoresce in relative proportion to the quantity of nucleic acid product produced. The TaqMan® procedure (Applied Biosystems, Calif.) describes one such fluorescent methodology for performing quantitative PCR.

Briefly described, the TaqMan® system integrates the use of a detectable reporter construct which comprises both a fluorescent label molecule and a quencher molecule. As long as the reporter construct remains intact, fluorescent label molecule emissions are absorbed by the quencher molecule. During the amplification process, however, the reporter construct is cleaved and the quencher molecule is released allowing the fluorescent label molecule emissions to be detected. The quantity or intensity of observed fluorescence may then be correlated with the amount of product formed throughout the reaction. Using this information, the initial quantity of target present in the reaction may be determined. Additional information describing the principles and applications of quantitative PCR can be found in: Real Time Quantitative PCR, Genome Research, Cold Spring Harbor Laboratory Press, 1996 and PCR Technology: Principles and Applications for DNA Amplification. Karl Drlica, John Wiley and Sons, 1997.

One characteristic feature of quantitative PCR-based amplification is that, the reaction kinetics typically change over the course of the reaction with the amount of product formed not necessarily increasing in a constant manner. For example, during the earlier cycles of a PCR reaction there may be an approximate doubling of the nucleotide strands with each cycle (exponential amplification). In the later cycles of the reaction, however, the efficiency of the amplification process may be diminished resulting in non-exponential amplification. Some of the factors that may affect the amplification efficiency include limiting quantities or depletion of reagents and competition for reaction products. The aforementioned changes in reaction kinetics may result in difficulties in determining the initial target concentration without performing detailed analysis of the reaction profile. In one aspect, it is desirable to monitor the reaction at various time or cycle intervals and acquire data which quantifies the emitted fluorescence of the reaction at these intervals. Using this information, data analysis methods may be used to assess the acquired fluorescence measurements and determine the initial concentration of target present in the reaction.

In quantitation methodologies, including real-time PCR, the fluorescence intensity for each amplification reaction may be determined using a charge-coupled device (i.e. CCD camera or detector) or other suitable instrument capable of detecting the emission spectra for the label molecules used in the reporter construct. Fluorescence samplings are performed over the course of the reaction and may be made at selected time intervals (for example: 25 millisecond samplings performed at 8.5-second intervals). In one aspect, emission spectra are measured for both the label molecule and the quencher molecule with the emission intensity resultant from the quencher molecule changing only slightly compared to that of the label molecule. The emission intensity of the quencher molecule may further be used as an internal standard to normalize emissions generated by the label molecule.

For each amplification reaction, the measured emission spectra obtained from the fluorescence samplings form an amplification data set that may be processed to determine the initial target concentration. In one aspect, the amplification data set comprises fluorescence intensity information obtained from a plurality of independent or coupled reactions. These reactions may be performed simultaneously or at different times wherein the data is accumulated and collectively analyzed. Furthermore, the amplification data set may further comprise fluorescence intensity data obtained from one or more standards whose initial target concentration is known.

In practice, the fluorescence signal generated during an amplification reaction may take on various characteristics associated with the chemical reactions involved and/or the instrumentation used to conduct/monitor the reaction. For example, it may be observed that gradual increases or decreases in signal level arise with increasing cycles. These signal level changes, however, may not necessarily be directly associated with the amplification of the target genetic material.

Additionally, the amplification profile for a selected reaction may reflect a sigmoid shape. In such instances, the increase in abundance of the target genetic material may slow and ultimately stop at some point due to chemical limitations. Furthermore, noise may be observed in the form of spikes or humps in the signal data. Such noise may be observed in earlier cycles, originating as high values followed by a decay to baseline. Noise may also take the form of Steps in the approximate middle of the signal, up and down excursions, weak growth-like signals, and other forms. In such instances, the observed noise may have nothing to do with the growth of the target genetic material. In certain instances, the growth/amplification rate may be significantly slower than theoretical doubling or observed amplification may be represented by very early growth, within four or five cycles.

As will be described in greater detail herein, aspects of the present teachings describe a novel approach for automatically establishing the amplification profile for a reaction in view of chemical and other limitations. Improving the interpretation of the amplification data also can enhance estimations of threshold cycle (which can be designated Ct, or Cₜ) values from real-time PCR data and their use in genetic analysis.

Overall, this approach may be helpful in addressing the aforementioned characteristics of the amplification data as well as increase the sensitivity and specificity of instrumentation such as the ABI PRISM 7000 (Applied Biosystems, Foster City Calif.) used in quantification assays. An exemplary software package used in connection with this instrument that may be configured to implement the disclosed analytical approaches is the “Sequence Quantification Software Package” (Applied Biosystems, Foster City Calif.). Additional details describing this package may be found in the User's Guide: Sequence Quantification Software v3.0 (PN: 5001194), and the User's Guide: Sequence Quantification Software Plus v1.0, each of which is incorporated by reference.

The methods described herein represent a potential advance over existing approaches improving performance concerning avoiding false positives and false negatives. Moreover, this facilitates a more reliable positive identification of bona fide growth and producing an estimated Ct value with low variability. In one aspect, false positives and false negatives may arise from inaccurate baseline determination. False positives may also arise from dye bleedover and/or crosstalk. In such circumstances, thresholds for determining Ct values may be set too high to capture signals of low-concentration samples. An advance over conventional approaches provided by the present teachings improves performance by accurately assessing the portion of the amplification signal that should be taken to be the baseline. Additionally, the present teachings may be used to distinguish crosstalk and/or bleedover from a bona fide amplification signal. The methods described also provide the ability to accommodate a wider range of amplification signals.

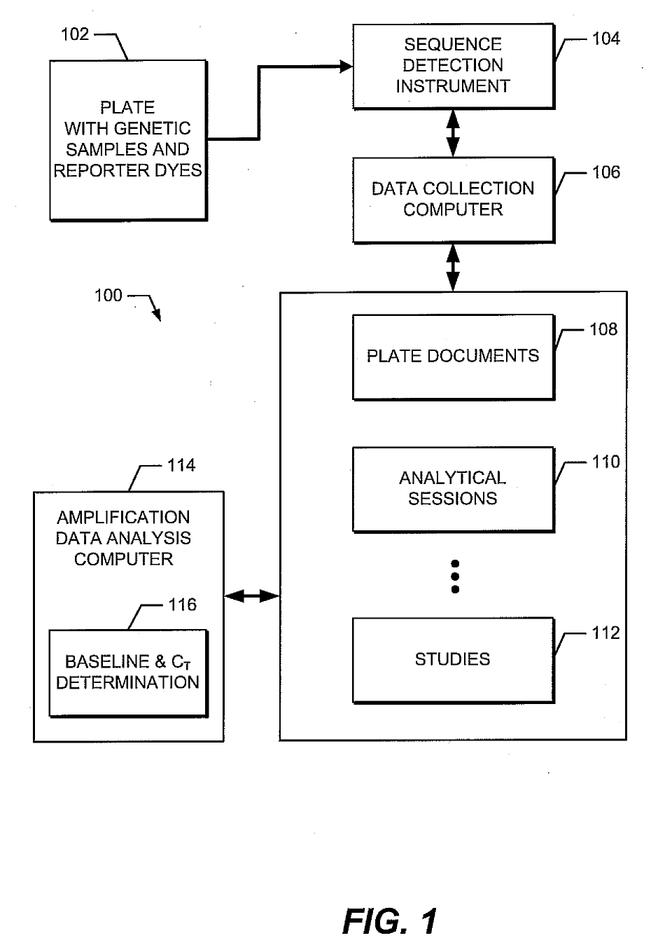

FIG. 1 is a schematic illustrating a system for spectral detection and analysis in accordance with some implementations of the present teachings. System 100 includes a plate 102 with genetic samples, a sequence detection instrument 104, a data collection computer 106, plate documents 108, analysis session 110, studies 112 containing analytical results from many plates and a amplification data analysis computer 114. To improve the quality of information being processed, sequence data analysis computer 114 further utilizes one or more baseline and Ct determination approaches 116 to automate the analysis of the data using information from the plate documents 108, analytical sessions 110, and the studies 112 that were obtained from the genetic samples in plate 102.

Sequence detection instrument 104 includes a spectral detector capable of distinguishing certain spectral species emitted from the fluorescence of reporter dyes interacting with the genetic material in wells on plate 102. The spectra are typically monitored in real-time as a thermal cycler in the sequence detection instrument 104 performs PCR on the genetic material. For example, PCR operations may cause the sample or target genetic material to replicate and hybridize with increasing amounts of a SYBR green dye detectable in the wells of plate 102. After several thermal cycles, the concentration of the target increases along with a detectable rapid increase of fluorescence from the SYBR green dye or other reaction substrate. A cycle threshold or Ct measurement is then identified when the measure of fluorescent intensity increases linearly on a logarithmic scale compared with the increasing cycle number. Subsequent analysis of Ct values among various reactions may be used to identify a concentration of the target genetic material.

Data collection computer 106 gathers raw data provided by sequence detection instrument 104 and stores in plate documents 108 as required by a particular study or experiment being performed. The raw data is labeled, organized and stored by data collection computer 106 in one of several different storage areas or files for subsequent processing. For example, the example in FIG. 1 depicts data collection computer 106 as capable of storing the raw data in as plate documents 108 or studies 112. In some cases, data collection computer 106 may also perform certain calibration operations or other types of basic data analysis with the results to be stored in analysis sessions 110.

Resulting data stored in plate documents 108, studies 112 and analysis sessions 110 are then made available to sequence data analysis computer 114. Operations in amplification data analysis computer 114 not only may perform baseline and Ct determination but also improves computational analysis associated with genetic analysis. In particular, aspects of the present teachings provide automated baseline and Ct determination operations 116 for increasing throughput of analysis while improving accuracy.

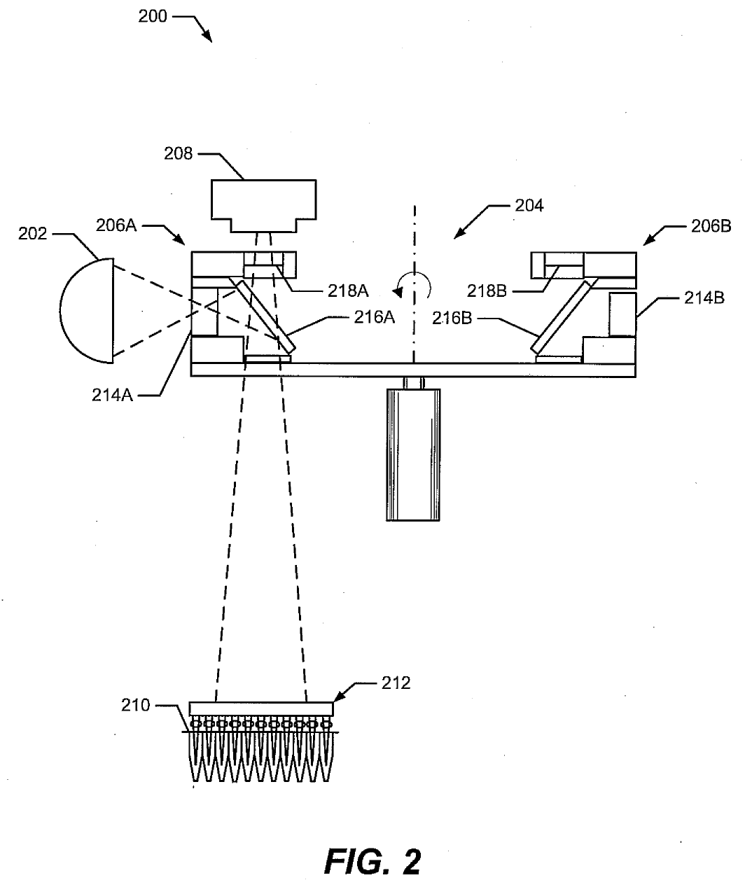

FIG. 2 is a schematic illustration of a system 200 used for fluorescent signal detection in accordance with implementations of the present teachings. This illustration depicts certain features typically associated with the Applied Biosystems 7500 Real-Time PCR System. However, aspects of the present teachings should not be limited by any one or several features associated with this equipment. Consequently, various aspects of the present teachings can be used in conjunction with the Applied Biosystems 7900HT Fast Real-Time PCR System model as well as almost any other device involved with gathering and/or analyzing spectra from a genetic sample.

Accordingly, detection system 200 illustrates some of the components making up spectral detector and optics in sequence detection instrument 104 previously described in FIG. 1. Detection system 200 can be used with real-time PCR (RT-PCR) processing in conjunction with aspects of the present teachings. As illustrated, detection system 200 includes a light source 202, a filter turret 204 with multiple filter cubes 206, a detector 208, a microwell tray 210 and well optics 212. A first filter cube 206A can include an excitation filter 214A, a beam splitter 216A and an emission filter 218A corresponding to one spectral species selected from a set of spectrally distinguishable species to be detected. A second filter cube 206B can include an excitation filter 214B, a beam splitter 216B and an emission filter 218B corresponding to another spectral species selected from the set of spectrally distinguishable species to be detected.

Light source 202 can be a laser device, Halogen Lamp, arc lamp, Organic LED, an LED lamp or other type of excitation source capable of emitting a spectra that interacts with spectral species to be detected by system 200. In this illustrated example, light source 202 emits a broad spectrum of light filtered by either excitation filter 214A or excitation filter 214B that passes through beam splitter 216A or beam splitter 216B and onto microwell tray 210 containing one or more spectral species. Further information on light sources and overall optical systems can found in U.S. Patent Application 20020192808 entitled “Instrument for Monitoring Polymerase Chain Reaction of DNA”, by Gambini et al. and 200438390 entitled “Optical Instrument Including Excitation Source” by Boege et al. and assigned to the assignee of the present case.

Light emitted from light source 202 can be filtered through excitation filter 214A, excitation filter 214B or other filters that correspond closely to the one or more spectral species. These spectrally distinguishable species may include one or more of FAM, SYBR Green, VIC, JOE, TAMRA, NED, CY-3, Texas Red, CY-5, Hex, ROX (passive reference) or any other fluorochromes that respond by emitting a detectable signal. In response to light source 202, the target spectral species and selected excitation filter, beamsplitter and emission filter combination provide the largest signal response while other spectral species with less signal in the bandpass region of the filters contribute less signal response. Multicomponent analysis is typically used to determine the concentration of the individual species according to their respective contribution to the emitted spectra.

Referring to FIG. 2, microwell tray 210 generally contains the genetic target sample with one or more reporter dyes corresponding to the assay used in conjunction with an experiment. Microwell tray 210 can include a single well or any number of wells however, typical sets include 96-wells, 384-wells and other well configurations. Of course, experiments may be designed to use many other plate configurations having different multiples of wells other than 96. The sample and particular combination of dyes used in the selected assay may be sealed in microwell tray 210 using heat and an adhesive film to ensure they do not evaporate or become contaminated.

Detector 208 receives the signal emitted from spectral species in microwell tray 210 in response to light passing through the aforementioned filters. Detector 208 can be any device capable of detecting fluorescent light emitted from multiple spectrally distinguishable species in the sample. For example, detector 208 can be selected from a set including a charge-coupled device (CCD), a charge induction device (CID), a set of photomultiplier tubes (PMT), photodiodes and a CMOS device. Information gathered by detector 208 can be processed in real-time in accordance with implementations of the present teachings and through subsequent post-processing operations.

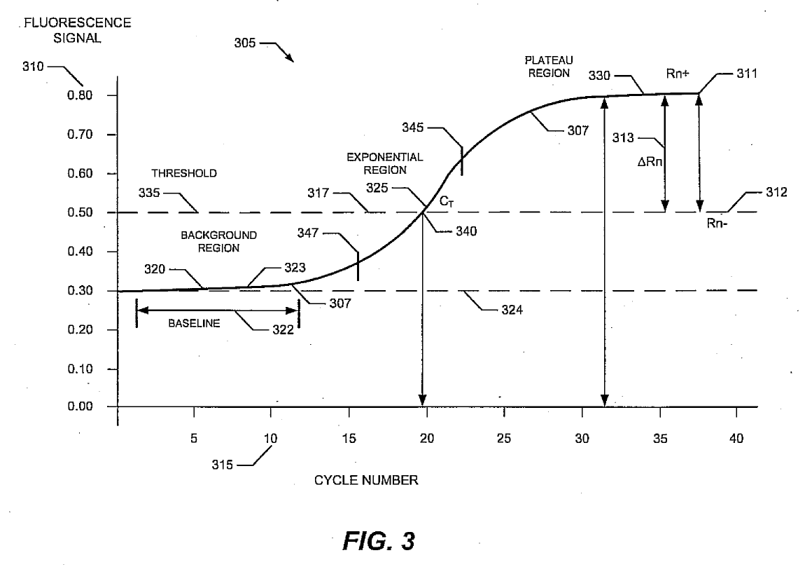

FIG. 3 illustrates an amplification plot 305 depicting the reaction characteristics for an exemplary nucleic acid target and the various analytical components that may be used to quantify the target. It will be appreciated that the amplification plot 305 is shown for the purposes of explanation and need not necessarily be constructed directly to apply the quantitative methods of the present teachings. However, the system can be configured to present a graphical representation of the amplification data set to aid a user in visualizing the results of the analysis.

The amplification plot 305 comprises a plurality of data points 307 forming an amplification profile 317 which is indicative of the measured intensity of signal generated by the label molecules within the amplification reaction. In the amplification plot 305, the y-axis values 310 correspond to observed signal intensities generated over the course of the amplification reaction. In one aspect, these signal intensities may correspond to fluorescent emissions obtained from instrumental sampling using a charge-coupled device or similar apparatus. Furthermore, the fluorescence detector may be configured to monitor wavelengths from approximately 500 to 650 nm. The x-axis values 315 correspond to the sample interval (shown as a function of cycle number) for the amplification reaction for which the signals are observed. Illustrated in this manner, the information represents the reaction progression as a function of the observed fluorescence intensities over the sampling interval and may be used to monitor the synthesis of progeny nucleic acid strands from an initial sample target.

When analyzing the amplification profile 317, various regions are identified and used in calculations for determining the initial concentration of target present in the reaction. Conventional genetic analysis methodologies generally require at least a degree of subjective interpretation. This subjective limitation often necessitates visually inspecting intensity data in order to identify these relevant regions of amplification profile 317. The effect of this subjective and somewhat manual approach to analysis may decrease the accuracy of quantitative analysis and their results, as well as, increase the analysis time.

In one aspect, the system and methods described herein overcome some of the limitations and drawbacks associated with conventional methodologies through the implementation of an analysis strategy that identifies significant regions of the amplification profile 317 in an objective and reproducible manner. As a result, aspects of the present teachings may improve the accuracy of quantification when determining the initial concentration of target present in an amplification reaction.

As shown by way of example in FIG. 3, the results from a typical quantitation reaction can be characterized by different regions including regions 320, 325, 330 within the amplification profile 317 corresponding to a background (noise) region 320, an exponential region 325, and a plateau region 330. During the earlier cycles of the reaction, the observed fluorescence produced by the label generally does not substantially exceed that produced by the quencher. Fluorescent emissions measured during these cycles are generally very low and may fall below the detection limits or sensitivity of the data acquisition instrumentation.

Furthermore, non-specific florescence arising from instrumental variations or noise within this background region 320 may significantly contribute to the observed signal. This may make it difficult to accurately determine the emission fluorescence arising from amplification in the early cycles of the reaction. Accordingly, implementations of the present teachings more accurately identify reaction fluorescence data falling in background region 320 to improve overall quantitation. For example, it may be desirable to accurately identify the range and bounds of the background region 320 so that this portion of the amplification reaction may be distinguished from exponential region 325 or plateau region 330 from amplification profile 317. Aspects of the present teachings contemplate that proper identification of background region 320 contributes to a more accurate measure of fluorescence in other regions and improved quantitation in other areas of the analytical process.

In one implementation, sub-region within the background region 320 may be identified as a baseline data set 322 and used in characterizing and analyzing background region 320. Baseline data set 322 serves as an indicator of the relative level of background fluorescence or noise from which exponential region 325 may be differentiated. As will be described in greater detail herein below, construction of the baseline 323 provides for the ability to quantify the relative noise present in the amplification reaction. Baseline 323 also can be used to normalize data points 307 of amplification profile 317 and partially compensate for the noise.

Exponential region 325 covers the region of amplification profile 317 that follows background region 320. It is within this portion of amplification profile 317 that the observed and measured intensity of fluorescence should increase exponentially (e.g. doubling sample concentration at each cycle). Within the exponential region 325, the detected quantity of fluorescence is typically sufficient to overcome noise that may predominate in the background region 320. The characteristics of the amplification reaction during the cycles associated with the exponential region 325 further reflect desirable reaction kinetics that can be used to perform quantitative target calculations. Together, both exponential region 325 and even plateau region 330 are sometimes referred to as part of “a growth region” since corresponding data points 307 generally exhibit a trend of substantially increasing or progressive fluorescence.

It will be appreciated that the increase in target concentration within the exponential region 325 need not necessarily follow a substantially exponential rate. Instead, this region 325 of the amplification profile 317 may be substantially characterized by a sub-exponential, geometric, linear and/or progressive rate of increase in target concentration. More generally, the amplification region 325 may be characterized as the portion of the amplification profile 317 where an increased rate of target accumulation may be observed relative to earlier and later cycles of the reaction. It will be appreciated that the methods described herein are suitable for assessing amplification reactions having a wide variety of characteristic increases in target concentration. For example, an increased rate of accumulation for a target should not be limited exclusively to assessing regions of “pure” exponential increase.

Delineation of discrete regions within the amplification profile 317 is useful for distinguishing characteristic reaction kinetics and further identifying portions of the amplification profile amenable to quantitation calculations. It will be appreciated by one of skill in the art that specific designation of these regions is not required to perform the quantitative calculations described herein.

It will further be appreciated that the characteristics of these regions may vary from one reaction to the next and may deviate significantly from illustrated profile. For example, in some amplification reactions, the exponential region 325 may extend over a different range of cycles and possess different intensity characteristics. Likewise, the background region 320 and the plateau region 330 may possess unique characteristics for each reaction. Additionally, other regions within the amplification profile 317 may be identifiable, for example, a region of substantial linearity may follow the exponential region 325. As will be described in greater detail hereinbelow, the quantitation methods may be desirably “tuned” or customized to accommodate potentially diverse classes of amplification profile characteristics.

The analytical approach used to quantitate the initial target concentration is based, in part, upon the identification of a threshold 335. In one aspect, the threshold 335 desirably aids in identifying and delineating noise present in the background region 320 and furthermore intersects with the amplification profile 317 at some point. The point of intersection between the threshold 335 and the amplification profile 317 is identified by a threshold cycle 340 or Ct 340. Ct (CT) represents the cycle number and fluorescence intensity when the amplitude profile 317 intersects with threshold 335. As will be appreciated by one skilled in the art, accurately determining Ct 340 is important as it likely influences subsequent calculations to predict the initial quantity or concentration of target present in the reaction. It may be noted that in embodiments, amplification profile 317 and any of its components can be or include a continuous signal, a discrete-time signal, a digitally encoded signal, a sampled or re-sampled signal, or other signal encodings or types.

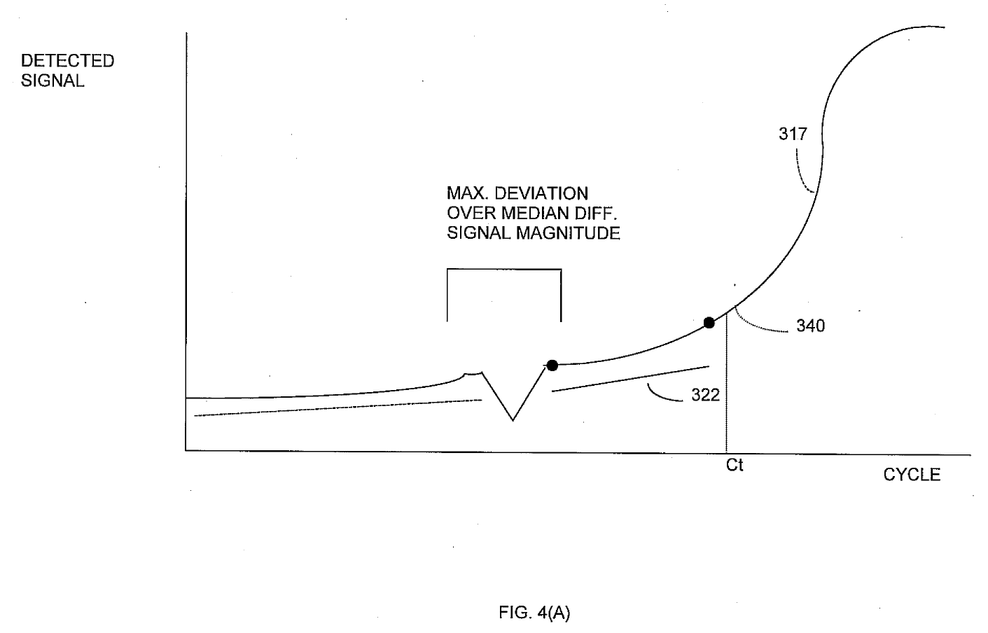

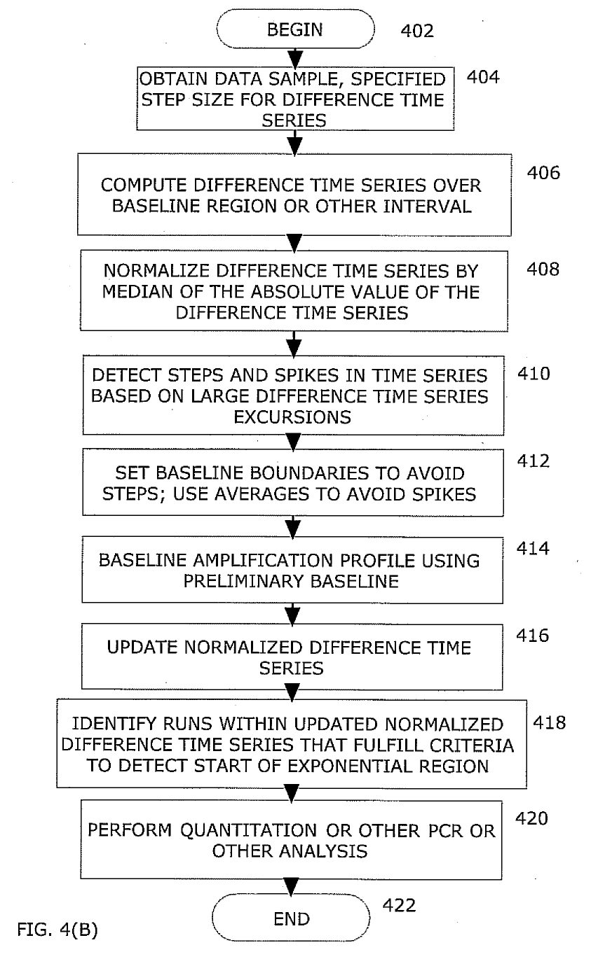

FIGS. 4(A)-7(B) illustrate diagrams providing details of approaches to techniques for signal analysis and noise identification, according to various embodiments of the present teachings. In embodiments, various signal processing and decision logic illustrated therein can be executed by a processor or controller embedded in a PCR or other instrument, by an attached or networked computer, or other processing resources.

FIGS. 4(A) and 4(B) illustrate aspects of methods for baseline analysis and noise detection in a detected signal obtained from a PCR or other reaction, according to various embodiments. As shown in FIG. 4(A), it is possible for a detected amplification profile to display anomalies early in the PCR cycle progression in which a rapid negative or positive transient spike occurs. In embodiments, a difference time series can be generated for the detected amplitude data points of an amplification data set to identify, isolate, or compensate for noise artifacts including sudden excursions of this class. In embodiments, the detected fluorescent or other signal amplitude can analyzed to determine a difference time series between the amplitude of detected signals at successive cycles, so that the amplitude at cycle 1 is subtracted from the amplitude at cycle 2, the amplitude at cycle 2 is subtracted from the amplitude at cycle 3, and so forth. In embodiments, rather than generating a difference time series based on successive or adjacent cycles, the difference time series can be generated using other pairs of cycle points that are not directly adjacent, but are separated by a step or “hop” as described herein. In embodiments employing a cycle hop, the separation between cycles can be hop sizes described herein, or can be pre-selected or designated to an appropriate or optimum size/value.

In embodiments, the difference time series can be generated for at least the baseline region of the amplification profile 317. In embodiments, the difference time series can be computed for a predetermined number of cycles, for all cycles, or for less than all cycles. The set of collective difference time series for all cycles under examination can, for example, be stored to local memory of a PCR instrument, or to an attached computer. In embodiments, at least two forms of the difference time series can be generated: 1) The median of the absolute value of the difference time series can be computed and the difference time series can be divided by this quantity at each point. This can be termed the normalized difference time series. 2) The median of the difference time series within the baseline interval can be computed, and subtracted from the difference time series. This can be termed the median deviate difference time series. The mean over the baseline interval of the absolute value of the median deviate difference time series can be computed and used to normalize the median deviate difference time series. This quantity can be termed the normalized median deviate difference time series. In embodiments, the normalized difference time series can be used, for example, to detect spikes, and/or to search for a large value in the signal followed immediately by a large value of opposite sign. The normalized median deviate difference time series can be used, for example, to detect Steps, and/or to search for a singular large value.

In embodiments, characteristics of the normalized difference time series can be used to determine which cycle or cycles encompass aberrations in the amplification profile. As shown in FIG. 4(A), the normalized difference time series showing a large value followed by another large value of opposite sign can correspond to the cycle at which a transient noise spike appears. This information can be used to normalize, adjust, or optimize the resulting baseline or other parameters. In embodiments, for instance, spikes, Steps, or exponential growth within an early portion of the amplification signal can be removed from adversely affecting baseline characterization.

In embodiments, the generation of the baseline signal can therefore compensate for the presence of that detected anomaly early in the amplification process. In embodiments, the baseline data set 322 (and/or baseline 323) can be shifted to a point after (rightward, at a later cycle) the normalized difference time series having the greatest amplitude, thus removing the transient spike from the baseline. In embodiments, rather than shift the baseline to a point downstream of the identified cycle displaying large normalized difference time series values, a substitution for the value at that cycle can be made. In embodiments, an average of detected signal values at adjacent cycles can be substituted for the signal value at that point. Other types of correction can be performed for the signal value at the cycle where the normalized difference time series displays large values. It may be noted that in embodiments, compensation on the foregoing basis can take into account more than one cycle point, so that, for example, the signals at the two (or more) cycles points where the normalized difference time series has large values can be identified, and the baseline data set 322 shifted or translated downstream of each identified cycle.

FIG. 4(B) illustrates overall signal processing on a normalized difference time series basis, according to various embodiments. In step 402, processing can begin. In step 404, a data sample can be obtained, for example from amplification profile 317, and a specified step (H) size can be obtained for generating a difference time series of the data sample. In step 406, a difference time series can be computed over the baseline region or other interval of amplification profile 317. In step 408, the difference time series can be normalized, for instance by dividing by the median of the absolute value of the difference time series. In step 410, signal artifacts or features, including for example Steps and spikes, can be detected in the time series based on the presence of larger difference time series excursions, for example those deviating by more than 50% over the normalized difference time series median or other measure.

In step 412, the boundaries of baseline data set 322 (and/or baseline 323) can be set to avoid Steps, and/or signal averages can be inserted or substituted to avoid or eliminate spikes. In step 414, the amplification profile 317 can be subjected to baselining using a preliminary baseline treated or generated by the preceding steps. In step 416, the normalized difference time series can be updated, for instance using the generated or updated baseline. In step 418, Runs within the updated normalized difference time series can be identified that fulfill criteria to detect the start of the exponential region of amplification profile 317. In embodiments, a Run can be defined as a set of successive values of the difference time series meeting a threshold criterion. Length for a Run can be a parameter in the algorithm. Criteria on a Run can consist of multiple thresholds for various points of the Run. For example, one criteria can be a requirement that two points of the Run exceed a first or comparatively high threshold while remaining points exceed a second, lower threshold. In step 420, quantitation or other PCR or other analysis can be performed using the detected start of the exponential region, or results or parameters. In step 422, processing can repeat, return to a prior processing point, jump to a further processing point, or end.

In embodiments, the normalized difference time series may be used to detect Steps in the amplification profile by looking for a large value. A start point may then be designated ahead of Steps so detected. In various embodiments, where the early points of the difference time series are generally large or whose value exceeds a designated threshold, the difference time series may be examined for indicators of early growth or clockwise rotation of the signal. This may be accomplished by reviewing the data for a Run of cycles where the magnitude of the difference time series exceeds a selected threshold and Run length exceeds a designated minimum value.

In certain embodiments, a Run that fulfills the selected criteria and has the maximum power determined, for example, as a summed amplitude over the length of the Run may be used in further analysis. Furthermore, a linear discriminant that is a function of the maximum power and start cycle of the Run may be used to detect early rising or increasing signals. A signal so detected may be associated with an early growth signal and the start cycle may be set to an early cycle such as one.

In various embodiments, a clockwise-rotated signal may be detected in instances such as where early growth of the signal is not observed. In such instances, a putative clockwise-rotated signal may be identified as potentially producing the large amplitude normalized difference time series in early cycles. This assessment may be further evaluated by checking that in the vicinity of the first and last cycles, the minimum normalized difference time series in these loci are large enough and the means of the normalized difference time series in these loci are nearly the same. If the criteria for this assessment have been met, a clockwise rotated signal may be identified as detected and the start cycle to set to one.

The analysis may further attempt to identify spikes or substantial or rapid changes in the difference time series. An exemplary spike may be observed as two substantially adjacent cycles having general characteristics such as: opposite polarity, large excursions, and/or nearly equal amplitude. Such spikes may be replaced or substituted with other signal data. In one embodiment, a spike may be substituted with data corresponding to an average of selected signal values at one or more neighboring points.

In various embodiments, an initial estimate may be used to reflect a preliminary assessment of the baseline zone. For example, the preliminary baseline zone may be designated as approximately 3-5 cycles from the start point to baseline the signal.

A growth sense (e.g. positive or negative) may further be established by examining the sign of the signal near the last cycle. This portion of the analysis may be performed following the aforementioned spike removal operation and preliminary baseline removal. In one aspect, performing the analysis in this manner desirably reduces the likelihood that the growth sense sign will be miscalculated as a result of single cycle noise.

Thereafter, a normalized difference time series may be computed for the data sample. During this analysis, a Run of the normalized difference time series may be identified that exceeds selected criterion to identify the start of the growth region. In one exemplary embodiment, a Run length with a minimum of approximately four may be selected. Values within this Run length may further be evaluated relative to one or more selected thresholds. For example, the normalized difference time series may have a condition imposed where approximately two values are expected to exceed a first/primary threshold and approximately all values are expected to exceed a secondary threshold.

Alternatively, or if the aforementioned analysis does not detect exponential growth, the preliminary baseline may be extended, such as by doubling the length of the preliminary baseline, and the analysis for normalized difference time series Runs repeated. In certain embodiments, where baseline extension does not provide a desired result, the analysis may flag a condition where the exponential growth region of the data sample cannot be determined using the normalized difference time series.

Using the designated baseline, the analysis may proceed by again attempting to identify Steps in the baseline. In various embodiments, Steps may be established by detecting large excursions in the median deviate difference time series over the baseline and verifying that this is not caused by an anomalously low value for the median of the normalized difference time series. Thereafter, the start point may be adjusted to follow the identified step if found.

In various embodiments, the baseline may be updated according to the following method. Initially, each signal may be scaled, for example, by performing a linear regression near or at the approximate middle region of each signal with the resulting value associate with the scale for each signal.

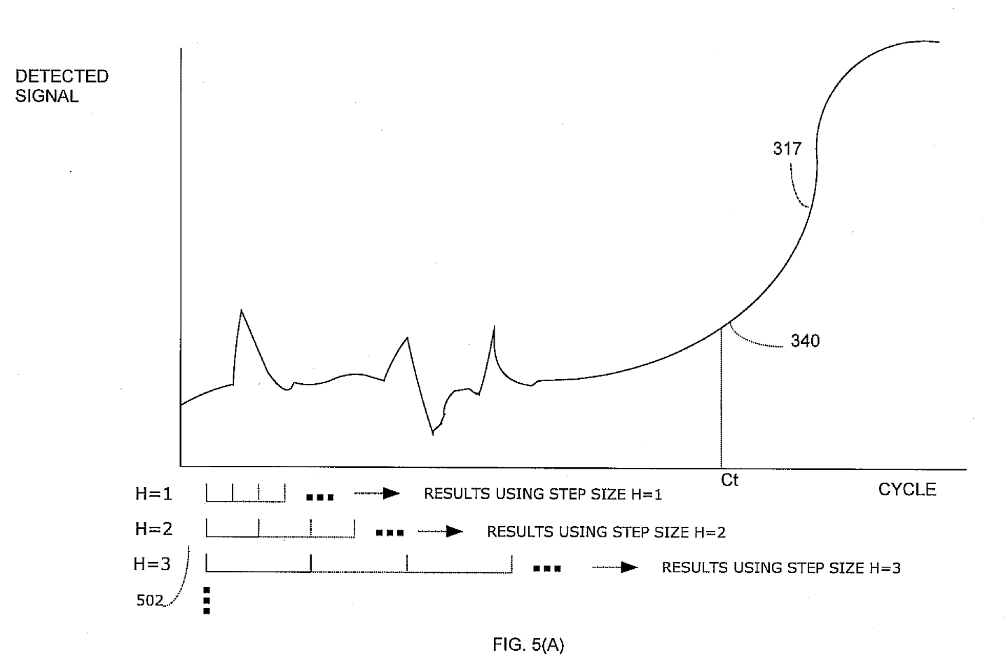

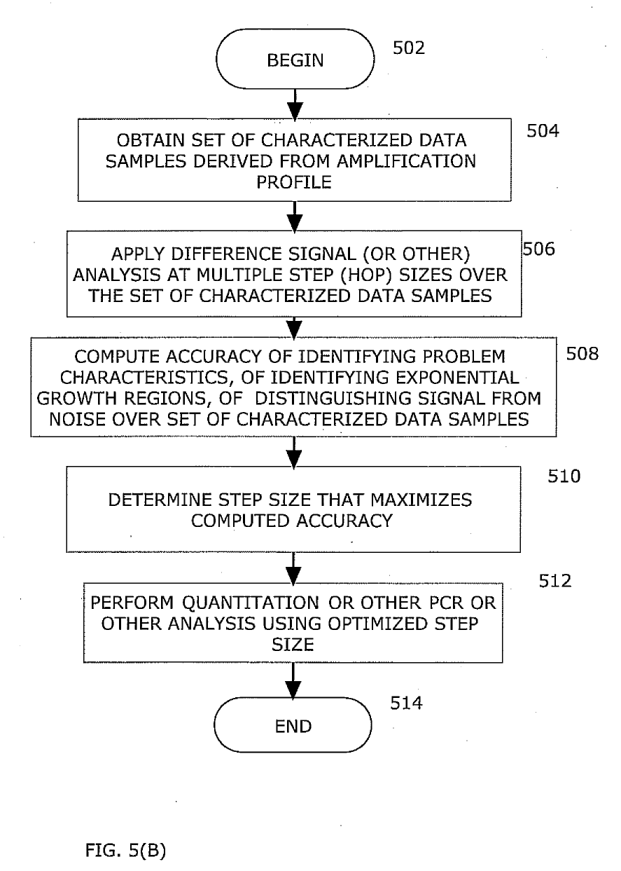

According to various embodiments, for example illustrated in FIGS. 5(A) and 5(B), a difference time series analysis or other noise detection procedure can be performed or extended by examining difference time series over several different “hop” or step sizes. For example, a difference time series using a hop size of 1 may be determined as the signal that results from subtracting the signal value at a first point from the signal value at the next point as the second point, the signal value at the second point from the value at a third point, etc. For a hop size of 2, the signal value of a first selected point may be subtracted from the signal value at a third selected point, the signal value at a second selected signal point subtracted from a signal value at a fourth point, and so forth for various step sizes. As shown in FIG. 5(A), for example, a set of step sizes 502 can thereby be generated which include a series of different “hop” sizes (H) or increments that, in one regard, generate or resample the data points into different-sized windows. In embodiments, the use of certain hop sizes can remove or omit transient noise spikes or other anomalies, thereby effectively smoothing the signal in the baseline region. In embodiments, a difference time series analysis as described herein can be conducted for each step size (H) in the set of step sizes 502, generating a corresponding set of results related to measured noise and other parameters for each individual step size.

In embodiments, the results of difference analysis or other noise analysis for each hop size (H) can be compared. In embodiments, the ensemble of difference analysis or other results collected over all step or hop sizes can be compared to develop a selected or optimized hop size. In embodiments, the step size within the set of step sizes 502 (H=1, 2, . . . n) that maximizes a desired quantity or criteria can be selected for analysis. In embodiments, the step size that generates the maximum accuracy in detecting anomalies in data samples (Spikes, Steps, etc.) and distinguishing signals from noise can be used to select a desired or optimized step size. Other noise detection thresholds, parameters, techniques, or criteria can be used to select a desired or optimized step size. In embodiments, for example, a signal slope measurement or computation, a signal power measurement or computation, or a curve fitting measurement or computation can be performed at the different step or hop sizes, to determine an optimized metric and corresponding optimized step or hop size.

Overall processing using a multi-resolution step or hop size technique is illustrated in FIG. 5(B). In step 502, processing can begin. In step 504, a set of characterized data samples derived from amplification profile 317 can be obtained. In step 506, a difference time series analysis or other analysis can be applied or generated at multiple step (or hop, H) sizes over the set of characterized data samples. In embodiments, the difference time series analysis can be or include the analysis described above in connection with FIGS. 4(A) and 4(B). In embodiments, the difference time series analysis can be applied using a step size H=1 to n.

In step 508, the accuracy of signal characteristics including characteristics of, or relating to, identifying problem characteristics, of identifying exponential growth region(s), and of distinguishing signal from noise can be computed or determined over the set of characterized data samples. In step 510, the step size that maximizes computed accuracy can be determined. In step 512, quantitation or other PCR or other analysis can be performed using the optimized step size. In step 514, processing can repeat, return to a prior processing point, jump to a further processing point, or end.

In various embodiments, for signals where growth was low or not detected as determined by the aforementioned difference time series analyses, the following processing can be applied (through paragraph [0077] below). A normalization process may be performed. In one aspect, the normalization process can be conducted by computing normalized partial slopes. The normalized partial slopes can comprise region slopes reflecting an early region near or around baseline endpoint, a middle region near or around approximately half of the amplitude point, and a late region near or around approximately the last cycle; where normalization further comprises dividing by the slope of approximately the maximum magnitude.

Thereafter, a y offset may be identified and removed from the signal starting from the approximate baseline start cycle and continuing onward for a selected portion of the signal. In various embodiments, where the signal has a positive sign value at or near the end a minimum value may be set to approximately zero. Alternatively, where the signal has a negative sign at or neat the end a maximum value may be set to approximately zero.

An initial estimate of baseline endpoint can then be generated. In one aspect, the baseline estimate can be determined as approximately the region where the signal data falls below a designated amount or fraction of the average amplitude around the approximate last cycle. A different fraction can be used dependent on whether or not there is observed signal growth. Growth can be presumed if the early partial slope has a value that is lower than a designated amount.

The baseline may again be updated and where a maximum amplitude at the approximate last cycle(s) does not exceed a selected number of standard deviations of the baseline, signal noise may be identified. Otherwise, the baseline end point may be evaluated to determine its locality to a spike. If the signal at the baseline end point differs from neighboring points by a threshold amount, a search may be performed between the baseline start point and the end point to identify a cycle at which the signal stays approximately below a selected threshold. Thereafter, the endpoint may be reset if such a cycle is identified or alternatively the end point may be set to a minimum distance from the start point if not identified thus reflecting a minimum selected baseline length.

The baseline may again be updated and for signals that have not been identified as noise, each candidate end point may be adjusted by a designated amount. (For example, to the left by a pre-selected amount). The baseline may again be updated and for signals where growth was not been detected in the difference time series analysis and have not been identified as noise further analysis performed. Here, if the maximum amplitude at the approximate end/last cycles does not exceed some designated number of standard deviations of the baseline signal, the signal may be identified as noise. Otherwise, the baseline endpoint may be designated as the point at which the signal starts differing from baseline by a preselected/designated number of standard deviations of the baseline. This analysis permits accounting for a low threshold that may contribute to bias of an earlier endpoint.

The baseline may again be updated and an iterative refinement of the start and end baseline cycles conducted. In one aspect, the end cycle may be determined by relocating the endpoint to a later cycle. For example, the end cycle may be moved until a selected number of points to right of the baseline end cycle consistently deviate from baseline by more than a designated number of the standard deviations of the baseline or the last cycle is reached. In one aspect, the start cycle may be determined by relocating the baseline start point to a later cycle. For example, the start cycle may be moved until the signal at the start point is within a selected number of standard deviations of the baseline.

The baseline may again be updated and an analysis made to detect a condition where there is a substantially short baseline in the approximate middle of a signal. In various embodiments, if the majority of the signal earlier than the start point is of opposite sign to most of signal later than the end, with a short baseline, the signal may be flagged a noise. A noise confirmation process may be conducted by evaluating at the difference time series. If the average difference before the baseline start point and after the baseline end point is relatively low and the difference between these two is also low, the signal may be confirmed as noise with the start point moved earlier to a point before where the difference time series exceeds a selected threshold.

During the analysis, a condition may be detected where there is a relatively short baseline and, following the baseline end point, there is data on the opposite side of the baseline with respect to growth sense. In such an instance, the baseline end point may be moved to a later cycle, for example, one cycle short of the point where the maximum opposite excursion occurs.

Thereafter the baseline may again be updated and a condition detected where there is a relatively short baseline, positive growth, and the data dips/falls below zero prior to increasing in value. This condition may be associated with the baseline end point being set too late in the time series. In the instance where there are two zero crossings, the baseline end point may be placed at the approximate midpoint between it and the first zero crossing. Thereafter the baseline may be updated and Z-score signals determined. In one aspect, Z-score analysis may be based on the approximate last estimate of the baseline end cycle (normalize by baseline variation). All signals may then be “flipped” as needed to reflect positive growth. For example, a signal may be multiplied by −1 if the signal is less than zero at the point where it reaches its maximum magnitude.

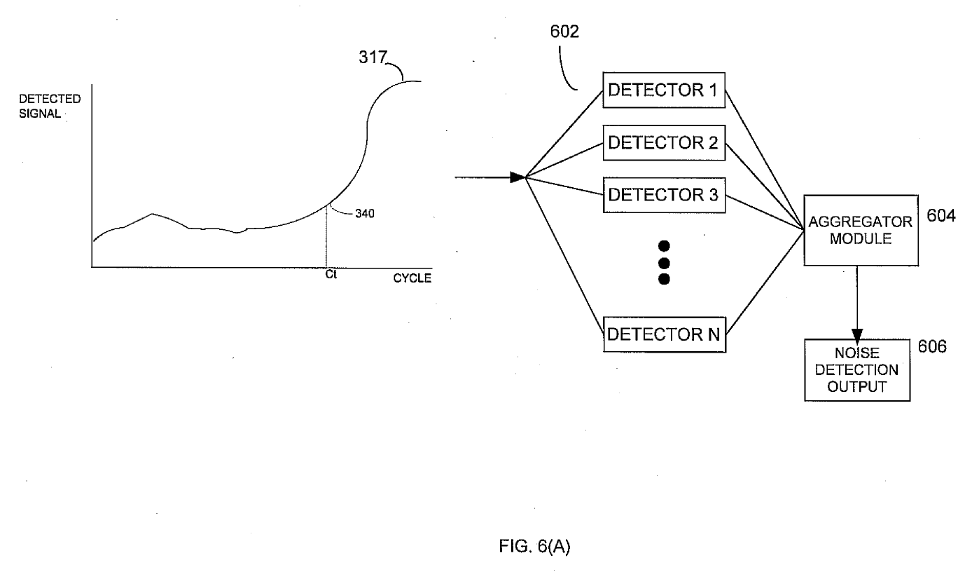

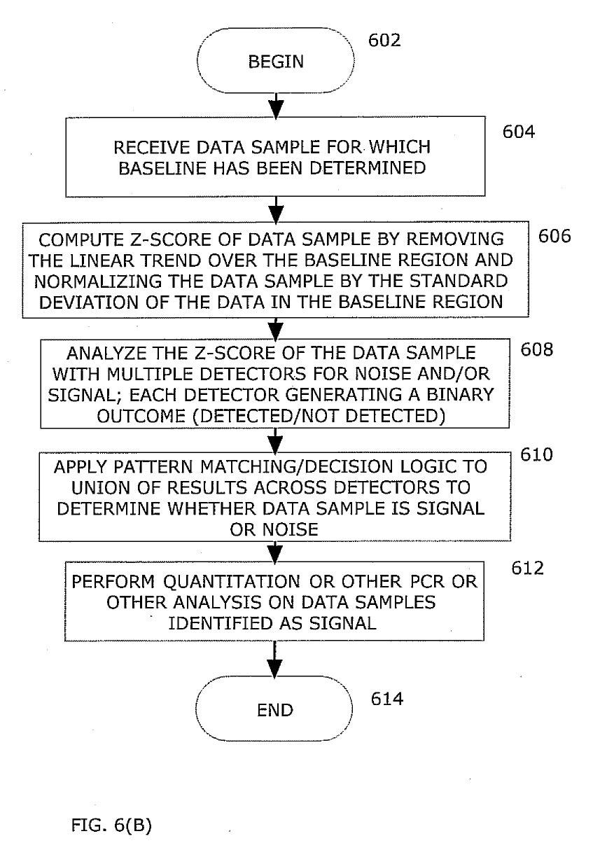

As illustrated in FIGS. 6(A) and 6(B), according to various embodiments of the present teachings noise detection in the amplification profile 317 can be performed using multiple noise detectors arranged in sets. The set of noise detectors can in general be applied to a portion or all of the amplification profile 317 to determine which of the discrete noise detectors is triggered or detects the presence of noise, according to the specific noise criteria or algorithm performed by that detector. An aggregator module 604 can then apply polling logic or other decision logic to the set of detection results produced by the set of noise detectors, to generate a decision whether noise is present, based on the combined determination of the set of noise detectors.

More particularly, as illustrated in FIG. 6(A), an amplification profile 317 can be transmitted to a set of noise detectors 602. The set of noise detectors 602 can comprise a plurality of software, hardware, or other logic units or modules each configured to conduct a discrete noise analysis on the supplied input signal. Exemplary routines, logic, and techniques that can be incorporated in individual noise detectors within the set of noise detectors 602 include:

i) Detecting a signal as noise during baselining operations. For example, a baselining analysis can be executed by techniques described above in which the baseline end point or cycle has been set at the last PCR or other amplification or other cycle, indicating that the baseline region has never been departed from.

ii) Determining that a Z-amplitude is lower than a selected threshold identifying possible noise. That is, in embodiments the mean of the of the amplification profile 317 over a portion or all cycles can be subtracted from the raw signal points in the baseline region or other region of the amplification profile 317, with the difference being divided by the standard deviation over the signal points over all cycles being examined.

iii) Evaluating the Z-amplitude as a function of the baseline end point. In embodiments, a linear discriminant may be used to determine the presence or onset of possible noise. In embodiments, a threshold can be set to decrease as the identified endpoint occurs in later cycles.

iv) Evaluating the slope of the baseline data set 322 (and/or baseline 323) and identifying as possible noise a deviation in slope that is substantially variant from the mean for baselines over the collective reaction, sample, or plate population. In embodiments, a slope value at signal point or points in amplification profile 317 that deviates by more than 2 standard deviations from the mean of the entire reaction, sample, or plate can be identified as noise. Other slope deviation thresholds can be used.

v) Noise can be identified if a majority of the signal earlier than the start point of baseline data set 322 (and/or baseline 323) is of opposite sign from a majority of signal later than the baseline end point. In embodiments, this noise identification criteria can be combined with a threshold baseline length corresponding to a relatively short baseline, for example, a threshold baseline length of 4 cycles, 5 cycles, or another number of cycles.

vi) In various embodiments, noise detection can be conditioned on partial slope data. Where the baseline end point is relatively early, or the signal is suspected of being noise and there is a short baseline in the middle of the data, the following operations can be performed: partial slopes (early, in the vicinity of baseline end point, middle, in the vicinity of the half amplitude point, late, in the vicinity of the last cycle) can be computed for the baseline data set 322 (and/or baseline 323). Noise can then be identified where the slope at the approximately half-amplitude cycle is below a selected threshold amount. If substantially no signal is detected by half-amplitude, an aspect ratio of the growth or exponential region can be evaluated. Noise can be identified if the aspect ratio is flat or below a designated slope. The aspect ratio can be evaluated based on a position where the aspect ratio resides between baseline end and last cycle. In embodiments, the aspect height (e.g. aspect ratio numerator) can further be normalized by delta Rn, or the difference between baseline end and last cycle. Finally, the aspect height normalized by cycle difference between baseline end and last cycle may be evaluated. Noise can be identified in the instance where the aspect ratio is below a selected value, and either of the other two aspect measures is also identified as low, or if both the aspect measures are determined to be low.

Noise can also be identified if the slope around the approximate baseline end relative to the other two partial slopes exceeds a selected amount or threshold, or where the minimum of the three partial slopes exceeds a selected amount or threshold.

vii) In embodiments, noise can be identified in conjunction with techniques for discriminating between noise and exponential growth using a second derivative analysis. In embodiments in this regard, growth detection can be based on second derivative analysis by recognizing that growth can be a function of the number of peaks in the second derivative analyzed together with a composite index of baseline length, Z-score time series amplitude, and positive curvature (as measured by the second derivative) of the Z-score time series. In one aspect, where the number of peaks in the second derivative exceeds a selected threshold amount it can be expected that the greater the index must be for growth to be identified. Conversely, noise can be identified where no growth is identified.

In various embodiments, the set of noise detectors 602 illustrated in FIG. 6(A) can be or include any one or more of the noise detectors illustratively enumerated in (i)-(vii) above. It will be appreciated that other numbers and types of noise detectors can be incorporated in the set of noise detectors 602. According to various embodiments, an aggregator module 604 can interrogate each of the individual noise detectors in the set of noise detectors 602, to generate a noise detection output 606. The noise detection output 606 can comprise an output indicating the presence of noise, the absence of noise, and indeterminate result regarding the presence or absence of noise, or other information.

Aggregator module 604 can apply decision logic to the results returned by individual noise detectors in the set of noise detectors 602, to produce noise detection output 606. In embodiments, the decision logic can include a voting or polling scheme in which each noise detector in the set of noise detectors 602 is interrogated to return a result. In such embodiments, the aggregator module 604 can count the number of detectors that are triggered to produce a positive detection of noise, versus those that do not. The total number of detectors identifying noise can be compared to a threshold or other decision criteria. In embodiments, for example, aggregator module 604 can be configured to generate a collective noise detection output 606 indicating noise where a selected or predetermined number of noise detectors are triggered/flagged, for example when 2 or more of the set of noise detectors 602 sense or detect noise. Other numbers of positive noise results or other criteria can be used. For example, aggregator module 604 can be configured to generate a noise detection output 606 indicating noise when more than half of the set of noise detectors 602 indicate noise. Other configurations of the set of noise detectors 602, aggregator module 604, and/or decision criteria can be used.

According to embodiments in further regards, for example, rather than a voting or polling scheme in which the results from each noise detector in set of noise detectors 602 is counted equally, the results from each noise detector can be assigned a weight or other scaling value. The results from individual noise detectors can therefore be given greater or lesser value in, or therefore make different contributions to, the resulting comparison or computation performed by aggregator module 604. In embodiments, for example, noise detector 1 of 7 can be assigned a weight of 0.9 while the remaining noise detectors can be assigned a weight of 0.1. If detector 1 returns a result or decision of noise being present, and aggregator module 604 is configured to generate a noise detection output 606 indicating noise when more than half of the noise detectors sense or identify noise, then aggregator module 604 will generator a positive finding of noise when detector 1 flags the presence of noise. This result is generated in that case because that detector's value contributes more than half the total, regardless of the results from the remaining detectors. Other weightings, scalings, ranges, and combinations of noise detectors and decision criteria can be used.

In embodiments in further regards, various selected combinations of noise detectors can be given special weighting or treatment, for instance when the detection by a pair or other grouping of noise detectors is known to have a particularly predictive effect. For example, where the number of noise detectors triggered or flagged in set of noise detectors 602 is 2, and the output of one of those detectors represents a failed second derivative growth test, noise may be identified in noise detection output 606, regardless of the results returned from other noise detectors. For further example, the triggering of a noise detector based on baseline slope deviation at the same time a noise detector based on z-scoring is triggered can be set to produce an automatic identification of noise in noise detection output 606, without regard to remaining noise detector results. Other combinations, types, weights, and decision logic criteria can be used.

Overall noise processing based on a multiple noise detector scheme is illustrated in FIG. 6(B). In step 602, processing can begin. In step 604, a data sample can be received for which a baseline data set 322 (and/or baseline 323) has been determined. In step 606, a Z-score of the data sample can be computed by removing the linear trend over the baseline region and normalizing the data sample by the standard deviation of the data in the baseline region. In step 608, the Z-score of the data sample can be analyzed with multiple detectors for noise and/or signal, with each detector generating a binary results or outcome (i.e., signal or noise detected/not detected). In step 610, pattern matching or decision logic can be applied to the collection or union of results generated across the set of detectors, to determine whether data sample represents signal or noise. In embodiments the ultimate decision or result can be rendered by aggregator module 604, and can be reflected in noise detection output 606.

In step 612, a quantitation analysis, or other PCR or other calculation, can be performed on data samples identified as signal. In step 614, processing can repeat, return to a prior processing point, jump to a further processing point, or end.

According to various embodiments, after this or other processing, the baseline data set 322 (and/or baseline 323) therefore can be generated or updated, and a Ct threshold determined. Thereafter, the Ct threshold may be used to locate an intersection of data with this threshold. In various embodiments, the threshold may be designated as a multiple of the average baseline variation across one or more samples, plates, or reactions. The intersection with the threshold may further be estimated using a cubic spline interpolation approach. In various embodiments, a Ct may not be called if the signal-to-noise ratio is falls below a designated amount where the signal-to-noise ratio is defined compares average amplitude at late cycles to the variation in the baseline interval.

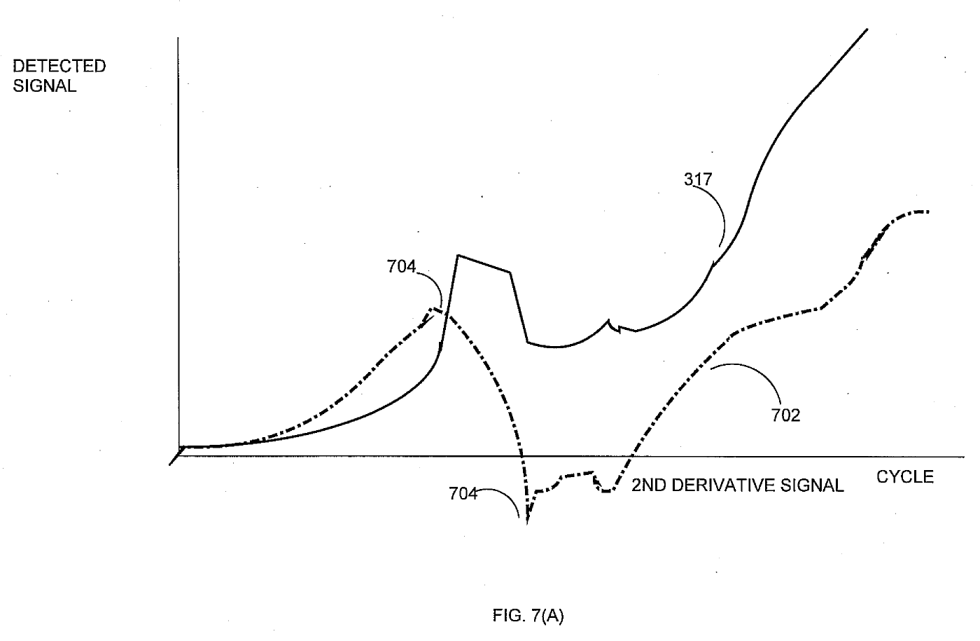

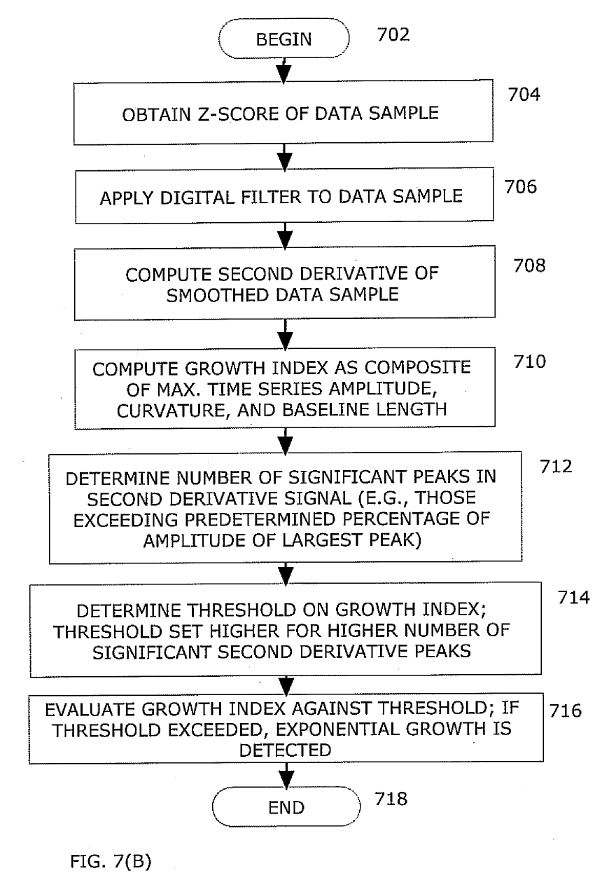

According to various embodiments illustrated in FIGS. 7(A) and 7(B), an analysis on amplification profile 317 can be performed to detect the onset of the exponential region or phase contained in that profile. In embodiments as shown in FIG. 7(A), a second derivative signal 704 of amplification profile 317 can be generated, generally indicating the rate of change of the rate of change in signal excursions in a baseline region or other portion or all of amplification profile 317. In general, a signal in the baseline region that contains a high degree of transient energy can display rapid changes in amplitude and/or direction, and those comparatively sharp changes produce a set of second derivative peaks 704 when the second derivative around those transients is computed. In embodiments, a total number of second derivative peaks can be computed for a baseline region or other portion or all of amplification profile 317. The total number of second derivative peaks can be used as a parameter to further characterize the amplification profile 317. According to embodiments described elsewhere herein, the total number of second derivative peaks can be used to adjust the stringency of criteria applied to identify noise.

In this regard, a greater number of second derivative peaks indicates a more sharply varying signal, so that sharper noise criteria should be applied. According to embodiments, the total number of second derivative peaks can be analyzed or generated together with a composite index based on other parameters of baseline data set 322 (and/or baseline 323). In embodiments, those embodiments can include baseline length (e.g. in total cycle length), maximum or substantially maximum signal amplitude (e.g. in z-scores), and maximum or substantially maximum positive curvature of amplification profile 317 located in the baseline region or elsewhere. In one aspect, where the number of peaks in the second derivative exceeds a selected threshold amount, for example, 3 or 4 peaks or another quantity, a sharper or more stringent set of noise criteria and/or detection criteria for the onset of exponential growth can be applied. For example, when the peak threshold amount is reached or exceeded, the composite index required for growth to be identified can be increased. Setting the composite index required for growth to a higher quantity can imply that one or more of baseline length, maximum signal amplitude, and maximum positive curvature, or their combination, reflect significantly higher values to reflect genuine or valid exponential growth. Again conversely, noise can be identified where no growth is identified based on the composite index adjusted for the number of second derivative peaks detected.

Overall second derivative processing according to various embodiments is illustrated in FIG. 7(B). In step 702, processing can begin. In step 704, a Z-score of a data sample can be obtained. In step 706, a digital filter can be applied to the data sample to smooth the data sample. In step 708, the second derivative of the smoothed data sample can be computed. In step 710, a growth index can be computed as a composite of a maximum time series amplitude, curvature values, including, for instance, a maximum positive curvature amount, and a baseline length. In step 712, the number of significant peaks in the second derivative signal can be determined. In embodiments, peaks can be identified when they exceed a predetermined percentage, for instance 70% or 80% or other value, of the largest peak. In step 714, a threshold on the growth index can be determined, the threshold being set or adjusted higher for a higher number of significant second derivative peaks. In step 716, the growth index can be evaluated against the threshold, and if the growth index exceeds the threshold, exponential growth can be detected or declared. In step 718, processing can repeat, return to a prior processing point, jump to a further processing point, or end.

The analysis approaches described herein aid in the processing of amplification or signal data so as to improve the correct or accurate handling of signals, including those containing signal anomalies. Signals containing anomalies can include signals which contain gradual Steps appearing in the baseline. In accordance with the present teachings, signals with relatively short baselines can be identified. Further, the analysis approaches can be configured to recognize signals where signal growth starts early in the amplification or substantially immediately during detection. The analysis approaches additionally permit that recognition of various cases of positive signals that may be subject to interpretation as being noise by conventional analysis approaches. The present teachings can additionally increase the accuracy of the analysis, especially in discerning signal from noise and improving the accuracy of determining appropriate intervals for the baseline region.

Having thus described various implementations and embodiments of the present teachings, it should be noted by those skilled in the art that the disclosures are exemplary only and that various other alternatives, configurations, adaptations and modifications may be made within the scope of the present teachings. For example, various implementations of the present teachings are described as being used for gene expression however, it is contemplated that the processing, analysis and graphical user interface described can be used directly for or adapted for use in genotyping data, allelic discrimination type studies as well as any other type of biological or genetic analysis.

Embodiments of the present teachings can be implemented in digital electronic circuitry, or in computer hardware, firmware, software, or in combinations thereof. Apparatus of the present teachings can be implemented in a computer program product tangibly embodied in a machine-readable storage device for execution by a programmable processor; and method steps of the present teachings can be performed by a programmable processor executing a program of instructions to perform functions of the present teachings by operating on input data and generating output. The present teachings can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs.

Further, in embodiments, components or resources described as singular can be combined, and components or resources described as separate or distributed can be combined. Thus, the present teachings are not limited to the specific embodiments described and illustrated above. Instead, the present teachings are construed according to the claims that follow and the full scope of their equivalents thereof.

What is claimed is:

1. A method of processing a signal, comprising:
receiving an amplification profile derived from an amplification reaction;
selecting signal data points in the amplification profile separated by a first step size to generate a first set of signal data points;
selecting signal data points in the amplification profile separated by at least a second step size to generate at least a second set of data points; and
generating a metric based on the first set of signal data points and the at least second set of data points; and
selecting an optimized step size based on the metric.

2. The method of claim 1, further comprising performing an analysis of the amplification profile using the optimized step size.

3. The method of claim 2, wherein the analysis of the amplification profile comprises quantitation of a biological sample.

4. The method of claim 1, wherein the metric comprises a metric based on at least one of a difference time series computed at the first step size and the at least second step size, a slope measurement computed at the first step size and the at least second step size, a signal power measurement computed at the first step size and the at least second step size, and a curve fitting computation computed at the first step size and the at least second step size.

5. The method of claim 1, wherein the first set of signal data points and the at least second set of second data points each comprise data points in a baseline region of the amplification profile.

6. The method of claim 1, wherein the first step size and the at least second step size each correspond to an integer number of cycles of the amplification reaction.

7. The method of claim 1, wherein the at least second step size comprises a plurality of additional step sizes, and the at least second set of data points comprises a plurality of corresponding sets of additional data points.

8. A method of processing a signal, comprising:
receiving an amplification profile derived from an amplification reaction;
generating a set of difference time series based on a set of signal data points in the amplification profile;
generating a median difference time series magnitude based on the set of difference time series;
generating a deviation value of each difference time series from the median difference time series magnitude; and
generating a baseline representation for a baseline region of the amplification profile based on the generated deviation values.

9. The method of claim 8, further comprising performing an analysis of the amplification profile using the generated baseline representation.

10. The method of claim 9, wherein the analysis of the amplification profile comprises quantitation of a biological sample.

11. The method of claim 8, wherein generating a baseline representation comprises shifting a baseline representation to omit the signal data point corresponding to a maximum deviation value.

12. The method of claim 8, wherein generating a baseline representation comprises altering a baseline representation to substitute an averaged signal data point value for the signal data point corresponding to a maximum deviation value.

13. The method of claim 8, wherein generating a set of difference time series comprises generating a set of normalized difference time series.

14. A method of processing a signal, comprising:
receiving an amplification profile derived from an amplification reaction;
generating an index used to identify an onset of an exponential growth region in the amplification profile;
generating a second derivative signal based on signal data points in the amplification profile;
determining a number of peaks in the second derivative signal; and
generating an update to the index based on the number of peaks in the second derivative signal.

15. The method of claim 14, further comprising performing an analysis of the amplification profile based on an exponential growth region identified using the updated index.

16. The method of claim 15, wherein the analysis of the amplification profile comprises quantitation of a biological sample.

17. The method of claim 14, wherein the signal data points comprise signal data points in a baseline region of the amplification profile.

18. The method of claim 17, wherein the index comprises a metric based on at least one of a maximum signal amplitude in the baseline region, a maximum positive curvature in the baseline region, and a length of the baseline region.

19. A method of processing a signal, comprising:
receiving an amplification profile derived from an amplification reaction;
communicating the amplification profile to a set of noise detectors, each of the set of noise detectors applying noise detection logic to the amplification profile;
receiving noise detection results from each noise detector of the set of noise detectors; and
generating a noise detection output based on the noise detection results.

20. The method of claim 19, further comprising generating a baseline representation for a baseline region of the amplification profile based on the noise detection output.

21. The method of claim 20, further comprising performing an analysis of the amplification profile using the baseline representation.

22. The method of claim 21, wherein the analysis of the amplification profile comprises quantitation of a biological sample.

23. The method of claim 19, wherein generating a noise detection output comprises determining whether a total number of the noise detection results returning an indication of noise exceeds a threshold.

24. The method of claim 23, wherein the threshold comprises one of a predetermined number, a predetermined ratio, and a predetermined combination of noise detectors returning an indication of noise.

25. The method of claim 19, further comprising weighting the noise detection results received from each of the noise detectors.
